(19) United States
(12) Patent Application Publication
(10) Pub. No.: US 2005/0015049 A1
(43) Pub. Date: Jan. 20, 2005

(54) Temporary tissue spacer and pretreatment balloon

(76) Inventors: Robert F. Rioux, Ashland, MA (US); George Bourne, Southborough, MA (US); David Danielsen, Westborough, MA (US); Barbara Bell, Sudbury, MA (US)

Correspondence Address: Patrick J. Fay, Esq.;FAY KAPLUN & MARCIN, LLP, Suite 702, 150 Broadway, New York, NY, 10038, US

(21) Appl. No.: 10/620,495

(22) Filed: Jul. 16, 2003

(51) Int. Cl. A61M 31/00

(52) U.S. Cl. 604/103.01

(57) ABSTRACT

A device for preventing closure of a surgically created resection cavity within tissues of the body comprises an insertion member having a distal end for insertion into a surgically created resection cavity and a proximal end which remains outside the resection cavity and a lumen extending between the proximal and distal ends and an inflatable member deployable from the distal end of the insertion member, an inner chamber of the inflatable member being fluidly coupled to the lumen to receive an inflation fluid therefrom so that, when the inflation fluid is supplied to the inflatable member, the inflatable member expands so that an outer surface of the inflatable member contacts the surrounding tissue and moves the surrounding tissue out of the resection cavity. A method of treating tissue surrounding a surgically created resection cavity, comprises the steps of, after a portion of tissue has been surgically removed to create a resection cavity, inserting a distal end of a catheter into the resection cavity and deploying an inflatable element at a desired location within the resection cavity from a distal portion of the catheter in combination with the steps of inflating the inflatable element to contact inner surfaces of the resection cavity and maintain tissue surrounding the resection cavity in a position substantially corresponding to a position of the tissue prior to the creation of the resection cavity to prevent closure of the resection cavity by healing processes during a recovery period and, after the recovery period, treating the tissue surrounding the resection cavity.

### BACKGROUND INFORMATION

Many medical procedures require the surgical formation and maintenance of a cavity within a patient's body. For example, the treatment of certain tumors may require a multi-faceted approach that includes a combination of surgery, radiation therapy and chemotherapy. In such an approach, after an initial surgical procedure has been performed to remove as much of a tumor as possible, radiation and chemotherapy are performed to kill remaining cancerous cells that could not be removed surgically. These remaining cancerous cells are usually concentrated in an area surrounding the site of the surgery, and can best be reached by inserting therapeutic materials directly into the surgery site, in close contact with the affected tissues.

In the case of radiation therapy, one of the more effective treatment methods is brachytherapy in which a source of radiation energy is placed within the body of the patient at the site of the removed tumor to substantially evenly treat the region that formerly surrounded the surgically removed tumor. In addition to or instead of radiation therapy, therapeutic chemical compounds may be used to kill cancerous cells located in the vicinity of a surgically removed tumor. Stents or other minimally invasive devices may be placed in the area to be treated to deliver the therapeutic compounds. Chemotherapy compounds may also be used systemically, to kill cancerous cells throughout the patient's body.

Several difficulties may arise in performing the procedures described above. Radiation therapy and chemotherapy often cannot be initiated immediately after completion of the surgery to remove the tumor as the patient requires time to recover from the surgery. However, during this recovery time, the healing process begins to close down the cavity formed during surgery. And, once the cavity has closed, it may be difficult to perform additional procedures such as those described above which rely on placing therapeutic materials in contact with an inner surface of the surgically created cavity.

### SUMMARY OF THE INVENTION

The present invention is directed to a device for preventing closure of a surgically created resection cavity within tissues of the body comprising an insertion member having a distal end for insertion into a surgically created resection cavity and a proximal end which remains outside the resection cavity and a lumen extending between the proximal and distal ends and an inflatable member deployable from the distal end of the insertion member, an inner chamber of the inflatable member being fluidly coupled to the lumen to receive an inflation fluid therefrom so that, when the inflation fluid is supplied to the inflatable member, the inflatable member expands so that an outer surface of the inflatable member contacts the surrounding tissue and moves the surrounding tissue out of the resection cavity.

The present invention is further directed to a method of treating tissue surrounding a surgically created resection cavity, comprising the steps of, after a portion of tissue has been surgically removed to create a resection cavity, inserting a distal end of a catheter into the resection cavity and deploying an inflatable element at a desired location within the resection cavity from a distal portion of the catheter in combination with the steps of inflating the inflatable element to contact inner surfaces of the resection cavity and maintain tissue surrounding the resection cavity in a position substantially corresponding to a position of the tissue prior to the creation of the resection cavity to prevent closure of the resection cavity by healing processes during a recovery period and, after the recovery period, treating the tissue surrounding the resection cavity.

### BRIEF DESCRIPTION OF DRAWINGS

FIG. 1 is a schematic diagram showing components of a brachytherapy apparatus;

FIG. 2 is a schematic diagram showing an embodiment of a tissue spacer and pre-treatment balloon according to the present invention; and

FIG. 3 is a schematic diagram showing a different embodiment of a tissue spacer and pre-treatment balloon according to the present invention.

### DETAILED DESCRIPTION

As described above, cancer treatment often relies on a multi-pronged approach with an initial surgical procedure followed by radiation and/or chemotherapy of the tissue surrounding the site of the surgery. Alternatively, radiation therapy may be carried out using a radioactive source located outside the body in close proximity to the affected area.

Internal radiation therapy has several important advantages over other methods of treatment for breast cancer. For example, this procedure places the radiation source inside the cavity created by the removal of the tumor (i.e., the lumpectomy or resection cavity). Thus, the radiation is targeted to the tissue which had been closest to the removed tumor and which is the area where cancer is most likely to recur or in which cancerous cells left behind after the surgery are most likely to be found. In addition, as the radiation in this procedure is delivered from within the cavity, the amount of radiation exposure to healthy tissues is less than that from external radiation therapy in which multiple beams of radiation pass from outside the body through healthy tissue to the location. This in turn reduces the potential for side effects of the treatment. In addition, the more targeted application of radiation permits application of stronger doses, so that the treatment regime can be completed in a shorter time—often in a matter of days.

For breast cancer, some conventional methods rely on multicatheter internal radiation therapy, in which a plurality of catheters are placed in the breast (up to 25 tubes), and a radioactive seed is placed in each catheter. According to embodiments of the present invention, a simpler radiation delivery method is used, which can be carried out on an outpatient basis over a few days.

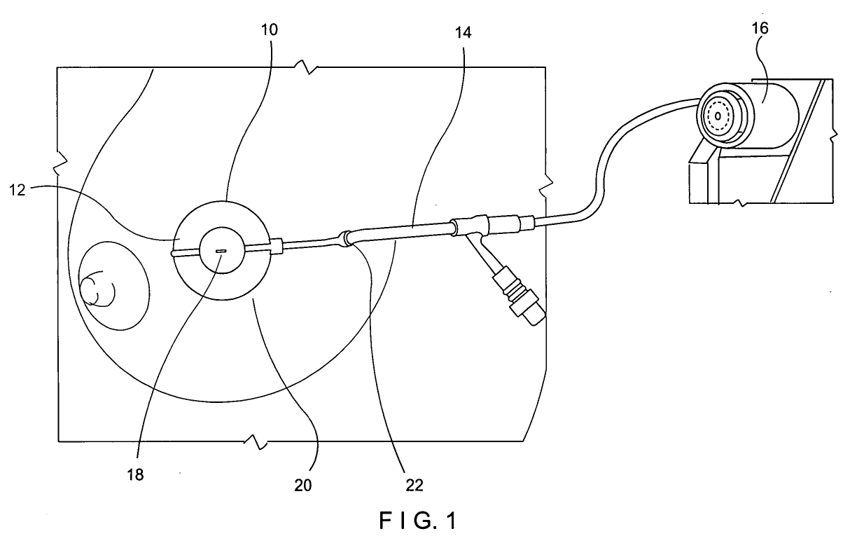

An exemplary method of delivering radiation therapy according to the present invention includes a balloon catheter that is inserted into a tumor resection cavity created by the surgical removal of a tumor. FIG. 1 shows an exemplary apparatus used to place the balloon catheter at a selected location, e.g., in the breast tissue. For example, the apparatus may include a MammoSite® RTS radiation therapy balloon produced by Proxima Therapeutics, Inc. As shown in FIG. 1, a balloon 12 is placed within the cavity 10 that is left in tissue 20 after a tumor has been surgically removed and the cavity 10 has been subjected to any post-lumpectomy treatment. The balloon 12 is placed via a catheter 14 which may be inserted to the resection cavity 10 from outside the patient's body through the incision made when the tumor was removed, or through a separate incision made with a scalpel at a later time. The balloon 12 is inserted into the resection cavity 10 in a deflated state and, once properly positioned is inflated using, for example, a saline solution and contrast media, to uniformly contact the tissue of the cavity so that the surrounding tissues and the inflated balloon 12 conform to one another.

The therapeutic effect of the radiation therapy balloon 12 is obtained by inserting thereinto a radioactive seed 22. For example, the seed 22 may be an Ir¹⁹² radioactive source. The seed 22 is connected by a wire to an afterloader 16, which is a computer controlled device that determines the exact location at which the seed 22 is to be placed within the balloon 12 and the length of time of the exposure, so that the appropriate amount of radiation is delivered to the targeted tissue. The afterloader 16 may be, for example, one of the devices manufactured by Nucletron, Varian and GammaMed HDR. As would be understood by those of skill in the art, the position of the seed 22 within the balloon 12 may be maintained by inserting a rod into the seed lumen. After the therapeutic session has been completed, the seed 22 is removed so that there is no source of radiation left in the patient's body between treatments. The balloon 12, however, may typically remain inflated within the cavity 10 between radiation sessions throughout the duration of the course of treatment. After the course of treatment has been completed, the balloon 12 is deflated and is removed together with catheter 14. The radiation therapy delivered with the balloon catheter may be used alone, or may provide a very targeted boost to other types of therapy, such as external beam radiation therapy and/or chemotherapy.

Certain difficulties may arise when treating tumors using balloon radiation therapy. Particularly in the context of breast conservation therapy (BCT) used to treat breast tumors with minimal resulting disfiguration, it may often be desirable to wait several days between the surgical removal of the tumor and the beginning of radiation treatments to attack any remaining cancerous cells. During this period the patient is allowed to recover from the surgery and regain her strength so that she can better withstand the side effects of radiation and/or chemotherapy. However, the healing process also takes place during this time, and begins to close the cavity left by the surgery. In fact, in as little as four days the cavity left by the removal of a breast tumor may close completely, and may disqualify the patient as a candidate for the radiation treatment balloon.

Embodiments of the present invention may be used to prevent the closing of the surgically created cavity before the patient is ready to receive additional treatment. The present invention thus lets the treating physician maintain a space of known size between the tissues of the patient, so that follow up treatment may be carried out at the appropriate time. In one embodiment according to the present invention, a pretreatment balloon is thus used as a temporary tissue spacer which is inserted in the cavity left by the removal of the tumor to prevent it from closing prematurely, and to maintain a defined space between the patient's tissues. The following description relates primarily to treatments for breast cancer, however, treatment of other types of cancer also may benefit from use of embodiment the pretreatment balloon and tissue spacer. In fact, those skilled in the art will recognize that such a tissue spacer may be valuable in any situation in which it is desired to maintain a cavity within the body and prevent the cavity from being closed by the healing process.

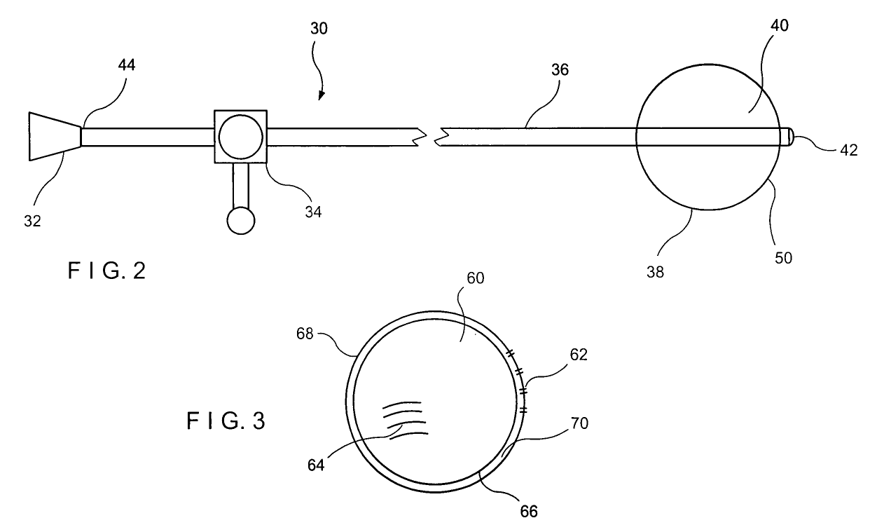

FIG. 2 shows an exemplary embodiment of a pretreatment balloon according to the present invention. This exemplary pretreatment balloon is used to temporarily separate tissues within the patient's body, to prevent the healing process from closing a resection cavity left by surgery to remove a tumor. As shown in FIG. 2, the pretreatment device 30 includes a catheter 36 having a distal end 42 which is insertable into the resection cavity, either through the incision made during the surgery or through a separate incision. A proximal end 44 is designed to remain outside of the patient's body, and may include various accessories to control and operate the balloon catheter. For example, an insertion/guidewire port 32 may be provided, to introduce a guidewire or other medical device into the lumen of the catheter 36. The port 32 or another port may be used to insert the deflated pretreatment balloon 40 into the catheter 36, and also to inject the fluid used to inflate the pretreatment balloon 40 that is deployed from the distal end 42 of catheter 36.

The catheter 36 may be sized to deliver a pretreatment balloon 40 of a desired diameter. Those skilled in the art will understand that, to minimize discomfort to the patient, it is desirable to use a catheter 36 having the smallest possible dimension compatible with the pretreatment balloon size. For example, the catheter 36 may have an outer diameter in the range of 5 to 10 FR or 0.066 to 0.131 inches, or more specifically, approximately 5 FR, or 0.066 inches. A catheter with such dimensions may be used, for example, with a guidewire with a diameter of 0.014 to 0.038 inches, or more specifically, about 0.014 in. In a different embodiment, a catheter 36 not requiring a guidewire may have a slightly smaller outer diameter. For example, the outer diameter of such a catheter 36 may be between 3 FR and 10 FR or, more specifically, approximately 4 FR, or 0.053 inches. A stopcock with a luer 34 may be used with a syringe to fill the balloon 40 with an inflation fluid, and to close the lumen of the catheter 36 to prevent the inflation fluid from leaking therefrom when the inflation has been completed. For example, the inflation fluid may be saline with a contrast media that facilitates observation of the pretreatment balloon 40 using fluoroscopy and/or ultrasound.

The pretreatment balloon 40 may preferably be sized to correspond to the size of the resection cavity and will consequently correspond to the size of the tumor that has been removed surgically. For example, a selection of balloons 40 having different diameters may be provided to the operating physician. The appropriate balloon 40 may then be selected to completely fill the cavity left by removal of the tumor, thus preventing the cavity from closing as the wound heals. Selecting the correct balloon size is important to prevent injury to the tissues surrounding the cavity that may occur if excessive pressure is placed on the surrounding tissue by a balloon 40 that is too large. In addition, if a balloon that is too small is selected, portions of the resection cavity may not be occupied thereby and may be prematurely sealed by the healing process. Alternatively, the amount of inflation of the balloon 40 may be controlled to obtain a desired maximum diameter of the pretreatment balloon 40 as long as the substantially spherical shape of the balloon 40 is maintained.

The pretreatment balloon 40 is designed so that, when inflated, it substantially fills the cavity left by the surgery. In addition, balloons 40 of various shapes may be provided so that a general shape of the balloon 40 may be selected which corresponds to the shape of the resection cavity. For example, a typical balloon 40 may, when inflated, have a diameter in the range of about 10 to 50 mm corresponding to the typical resection cavity size for certain procedures. However, those skilled in the art will understand that balloons in a wide range of sizes may be provided to be employed in cavities of various sizes and shapes. In one example, the pretreatment balloon 40 may have a substantially spherical shape, so that, after removal of a substantially spherical portion of tissue, all the tissue surrounding the resection cavity can be treated substantially equally effectively as the balloon 40 will substantially contact surrounding tissue along its entire outer surface 38. The pretreatment balloon 40 is inserted through the balloon catheter 36 to a desired position in the body in the deflated state. The balloon 40 is inflated after it has been removed from the distal end 42 of the catheter 36 so that it assumes its desired position in the resection cavity. Until the time when further radiation therapy treatment is to be carried out, the pretreatment balloon 40 acts as a tissue spacer, which prevents the resection cavity from closing and the tissues thereof from healing together.

In an exemplary embodiment according to the invention, the pretreatment balloon 40 may be coated with a therapeutical compound that assists in the treatment of the surrounding tissue. For example, a chemotherapy compound may be delivered via the pretreatment balloon 40, for example by being diffused through an outer surface thereof or by being coated on the outer surface thereof. For example, one compound that may be used in conjunction with the pretreatment balloon 40 is paclitaxel. Paclitaxel makes tumors more sensitive to being killed by radiation, and thus may allow the treatment to take place with a reduced dosage of radiation to achieve a desired clinical result, and to minimize the growth of diseased tissues.

Slow release doses of paclitaxel may be used on balloons 40 for the treatment of a variety of illnesses including tumors of the ovaries, the breasts, the lungs, the pancreas and the stomach. As the drug tends to make tumors much more susceptible to being killed by radiation, it is well suited for combination with a radiation treatment regime as described above.

In one exemplary embodiment, the pretreatment device 30 according to the present invention includes a pretreatment balloon 40 having an outer surface 38 that includes features adapted to dispense a therapeutic compound, such as paclitaxel, as part of a treatment for cancer. For example, polymeric carriers may be used as drug delivery vehicles to time-release the therapeutic agent. In balloons 40 according to this embodiment, the drug is embedded in a polymeric carrier which is coated on the balloon 40. The polymeric carrier degrades when in contact with body tissues and fluids, to progressively release the therapeutic agent to the tissues with which the balloon 40 is in contact. The rate of release of the therapeutic agent from the balloon 40 may be controlled by adjusting the composition of the polymeric carrier to achieve a desired concentration of the therapeutic agent across the outer surface of the balloon 40. Alternatively, the concentration of the therapeutic agent may be varied across the surface of the balloon 40 so that increased dosages of the agent may be applied to selected portions of tissue by orienting the balloon 40 so that the selected portions of tissue are in contact with these areas of increased agent concentration.

This method of agent delivery reduces the unwanted distribution of the drug to parts of the body which are not being targeted for such treatment, thereby lessening the risk of potentially harmful side effects. This added efficiency with lessened risk of side effects may reduced the number of treatments required or increase the efficacy of treatment by allowing more intense treatment of the targeted tissue.

In the exemplary embodiment shown in FIG. 2, the pretreatment balloon 40 has an outer surface 38 to which a layer 50 is applied to encapsulate one or more of a variety of therapeutic agents for release at a desired rate. According to the invention, the layer 50 may be made from a material that can be formed in different shapes and consistencies, such as nanospheres, microspheres, pastes, sprays, meshes and coatings. In the exemplary embodiment shown in the drawings, the layer 50 is formed of a polymeric material which forms a coating or a mesh on the outer surface 38 of the pretreatment balloon 40. The layer 50 includes a selected dose of one or more therapeutic agents such as, for example, paclitaxel, which agent(s) is (are) time released over a prescribed period into the patient. As described above, when the pretreatment balloon 40 is inflated within the resection cavity, the layer 50 is in direct contact with the tissue that is at the highest risk of recurrence. Thus, lower dosage amounts to the patient may achieve increased results with a decreased risk of side effects.

An exemplary procedure for using the pretreatment balloon to treat breast cancer, according to the present invention, begins with a conventional lumpectomy procedure performed to remove a tumor. After the lumpectomy has been completed, an uninflated pretreatment balloon such as that shown as balloon 40 in FIG. 2 is inserted into the tumor resection cavity. An applicator such as the catheter body 36 may be used to direct and place the pretreatment balloon 40 in the cavity. The catheter body 36 may include a luer 34 and a port 32 that remain outside the patient's body during the procedure. The port 32 may be used to insert the deflated balloon 40 into the catheter body 36. The distal end of the catheter body 36 is then placed in a desired location within the resection cavity for the deployment of the balloon 40 and the balloon 40 is ejected from the catheter body 36 into the resection cavity. When the pretreatment balloon 40 is in place within the cavity, the luer 34 may be used to fill the balloon 40 with saline solution and contrast media. The balloon 40 is filled to fit the edges of cavity 10, as shown in FIG. 1. The pretreatment balloon 40 may preferably be formed integrally with the catheter body 36 (e.g., by co-molding). Thus, the saline solution or other fluid within the pretreatment balloon 40 remains therein after the stopcock connected to the luer 34 has been closed. At this point the wound is cleaned and dressed, and the patient may leave the hospital until regaining sufficient strength to receive additional treatments.

During the patient's recovery time, the layer 50 releases the therapeutic agent(s) stored therein. In the case of paclitaxel, the targeted time release dose of the drug inhibits the survival of cancerous cells that may still exist in the tissue adjacent to the tumor that has been removed. As described above, the remaining cancerous cells also become more sensitive to subsequent radiation treatments by exposure to paclitaxel. In addition to a therapeutic agent such as paclitaxel, the layer 50 may include antibacterial compounds or other types of therapeutic agents suitable for the procedure. The layer 50 may preferably be designed to provide a substantially uniform drug coverage on the surface of the pretreatment balloon 40, and is preferably adapted for use on a flexible, inflatable surface without cracking, flaking or delaminating of the polymer and the drug contained therein. Both the layer 50 and the pretreatment balloon 40 are hypoallergenic and are bio-compatible, since they may be left in place within the patient's tissues for extended periods of time. Those skilled in the art will understand that, alternatively, concentration of the therapeutic agents may be varied across the surface 38 of the pretreatment balloon 40 if it is desired to apply higher dosages of therapeutic agents to certain portions of tissue than others.

When sufficiently recovered to resume treatment, the patient returns to a medical facility, preferably on an outpatient basis. Depending on the specific design of the pretreatment balloon 40, radiation therapy treatment may be carried out using the pretreatment balloon 40 itself as a container for a radioactive seed 22, as described above. That is, after a predetermined healing time has elapsed, a radioactive seed 22 may be inserted therein to begin radiation therapy. If there is no time lapse between the surgery and the initiation of the radiation treatment, the tissue spacing function of the balloon is not needed for the initial placement of the radioactive seed 22. Alternatively, after the pretreatment balloon 40 has been used to prevent premature healing by separating the surrounding tissue, it may be removed and a different radiation therapy balloon may be placed in the resection cavity to receive the radioactive seed 22. In cases where the pretreatment balloon 40 remains in use to carry out radiation therapy, a radioactive seed 22 attached to an external afterloader with a wire is inserted through a port 32 and is placed at a desired location (e.g., under computer control) within the balloon 40 so that the seed 22 resides at a desired position within the resection cavity. When the appropriate dose of radiation has been administered, the radioactive seed 22 is removed. If, after a particular application, the course of treatment has not yet been completed, the pretreatment balloon 40 is left inflated in the cavity, to prevent its closing until the next treatment session. Otherwise, the balloon 40 may be removed to allow the resection cavity to heal.

If the pretreatment balloon 40 implanted in the patient after surgery is not suitable to carry out balloon radiation therapy, additional steps may be carried out. For example, when the patient has sufficiently recovered and a course of post-surgery treatment is to be begun, the pretreatment balloon 40 may be deflated and removed from the resection cavity through the catheter body 36 and discarded. Then a radiation therapy balloon suitable for the intended course of treatment may be inserted in its place, as described above.

The balloon 40 may be deflated, for example by draining the inflating fluid through the catheter body 36 using the luer 34. After the balloon 40 is completely deflated, the balloon 40 may be drawn into the catheter body 36 and withdrawn from the body therethrough. The catheter 36 is then also removed. This removal procedure is preferably an outpatient procedure that should not require general anesthesia to be used. To complete the treatment, the wound left by catheter body 36 is closed and dressed. The patient may then be allowed to leave the medical facility without further delays or procedures.

In a different exemplary embodiment according to the present invention shown in FIG. 3, a therapeutic agent is dispensed to the patient through sideholes in the apparatus rather than through a polymeric coating disposed on the pretreatment balloon. In this case, an outer surface 68 of a pretreatment balloon 60 may be permeable to the therapeutic agent(s) to be dispensed to the patient while the inflating fluid remains sealed within the balloon 40. For example, the outer surface 68 may include perforations 62 that extend between a radially outer area 70 containing the therapeutic agent(s) and the exterior of the balloon, as shown in FIG. 3. An impermeable inner tube partition 66 may separate the radially outer area 70 from an inner area 64 in which the inflating fluid is located. In this manner, only the therapeutic agent in the radially outer area 70 can exit through the perforations 62 to reach the patient's tissues while the inflating fluid is maintained within the inner area 64.

The present invention has been described with reference to specific exemplary embodiments. Those skilled in the art will understand that changes may be made in details, particularly in matters of shape, size, material and arrangement of parts. Accordingly, various modifications and changes may be made to the embodiments without departing from the broadest scope of the invention as set forth in the claims that follow. The specifications and drawings are, therefore, to be regarded in an illustrative rather than a restrictive sense.

1. A device for preventing closure of a surgically created resection cavity within tissues of the body comprising:
an insertion member having a distal end for insertion into a surgically created resection cavity and a proximal end which remains outside the resection cavity and a lumen extending between the proximal and distal ends; and
an inflatable member deployable from the distal end of the insertion member, an inner chamber of the inflatable member being fluidly coupled to the lumen to receive an inflation fluid therefrom so that, when the inflation fluid is supplied to the inflatable member, the inflatable member expands so that an outer surface of the inflatable member contacts the surrounding tissue and moves the surrounding tissue out of the resection cavity.

2. The device according to claim 1, wherein the insertion member is a balloon catheter.

3. The device according to claim 1, wherein the inflatable member is a balloon insertable through the insertion member in a deflated configuration.

4. The device according to claim 1, further comprising a luer at the proximal end adapted to introduce inflation fluid to the inflatable portion via the lumen.

5. The device according to claim 1, further comprising a port at the proximal end adapted to introduce a radioactive seed into the inflatable portion via the insertion member.

6. The device according to claim 1, wherein the inflatable member further comprises a retention layer formed on the outer surface to retain a therapeutic agent thereon and dispense the therapeutic agent at a selected rate.

7. The device according to claim 6, wherein the retention layer is a polymeric coating.

8. The device according to claim 6, wherein the therapeutic agent is paclitaxel.

9. The device according to claim 1, wherein the inflatable member is a substantially spherical balloon.

10. The device according to claim 1, wherein the inflatable member further comprises a plurality of perforations in at least a portion of the outer surface so that a therapeutic agent may pass through the perforations to an outside of the inflatable member.

11. The device according claim 11, wherein the inflatable member comprises an inner inflation fluid chamber and an outer therapeutic agent chamber and wherein the inflation fluid chamber and the therapeutic agent chambers are sealed from one another, the perforations communicating the therapeutic agent chamber to an exterior of the inflation member.

12. A surgical tissue separator comprising:
an expandable portion insertable in a surgically created body cavity; and
a catheter portion adapted to position the expandable portion in the cavity, the expandable portion being deployable from the catheter portion;
wherein the expandable portion expands to contact inner surfaces of the cavity to prevent the inner surfaces from healing together.

13. The tissue separator according to claim 12, further comprising a lumen of the catheter portion connected to the expandable portion.

14. The tissue separator according to claim 12, wherein the expandable portion is an inflatable balloon.

15. The tissue separator according to claim 12, wherein an inflation fluid flows through the catheter portion into the expandable portion.

16. The tissue separator according to claim 12, further comprising a coating of the expandable portion adapted to time release a therapeutic compound to tissue surrounding the expandable portion.

17. The tissue separator according to claim 16, wherein the therapeutic compound includes a chemo-therapeutic agent.

18. The tissue separator according to claim 17, wherein the chemo-therapeutic agent comprises paclitaxel.

19. The tissue separator according to claim 16, wherein the coating is a polymeric coating adapted to encapsulate the therapeutic compound.

20. The tissue separator according to claim 12, wherein the expandable portion is a radiation therapy balloon.

21. The tissue separator according to claim 20, wherein the balloon is adapted to receive a radioactive seed therein through the catheter portion.

22. The tissue separator according to claim 12, wherein the expandable portion is substantially spherical.

23. The tissue separator according to claim 12, wherein the expandable portion is sized to fill a lumpectomy resection cavity.

24. A method of treating tissue surrounding a surgically created resection cavity, comprising the steps of:
after a portion of tissue has been surgically removed to create a resection cavity, inserting a distal end of a catheter into the resection cavity;
deploying an inflatable element at a desired location within the resection cavity from a distal portion of the catheter;
inflating the inflatable element to contact inner surfaces of the resection cavity and maintain tissue surrounding the resection cavity in a position substantially corresponding to a position of the tissue prior to the creation of the resection cavity to prevent closure of the resection cavity by healing processes during a recovery period; and
after the recovery period, treating the tissue surrounding the resection cavity.

25. The method according to claim 24, further comprising providing a time released therapeutic agent to at least a portion of the tissue surrounding the resection cavity during the recovery period.

26. The method according to claim 25, wherein the therapeutic agent is provided via the inflatable element.

27. The method according to claim 26, wherein the therapeutic agent is time released from a coating formed on an outer surface of the inflatable element.

28. The method according to claim 25, wherein the therapeutic agent includes paclitaxel.

29. The method according to claim 24, wherein, after the recovery period, the tissue surrounding the resection cavity is treated with radiation therapy by inserting a radioactive seed into the inflatable element.

30. The method according to claim 24, wherein the inflatable member is inflated by the introduction of at least one of saline solution and contrast media thereinto.
